(19) United States
(12) Patent Application Publication
(10) Pub. No.: US 2022/0411395 A1
(43) Pub. Date: Dec. 29, 2022

(54) PROCESS TO CONTINUOUSLY PREPARE A CYCLIC CARBONATE

(71) Applicant: NEW GREEN WORLD B.V., Gouda (NL)

(72) Inventors: Frank VERGUNST, Gouda (NL); Sander LAURENTIUS, Capelle aan den IJssel (NL)

(73) Assignee: NEW GREEN WORLD B.V., Gouda (NL)

(21) Appl. No.: 17/774,263

(22) PCT Filed: Nov. 30, 2020
(86) PCT No.: PCT/EP2020/081896

§ 371 Date: May 4, 2022

(30) Foreign Application Priority Data
Nov. 15, 2019 (NL) 2024242

(51) Int. Cl. C07D 317/36; C07F 5/06; B01J 31/22

(52) U.S. Cl. CPC C07D 317/36; C07F 5/069; B01J 31/2217; B01J 2531/31; B01J 2231/48

(57) ABSTRACT

The invention is directed to a process to continuously prepare a cyclic carbonate product by reacting an epoxide compound with carbon dioxide in the presence of a supported dimeric aluminium salen complex. The process is performed in a reactor comprising a slurry of the supported dimeric aluminium salen complex and liquid cyclic carbonate product. The produced cyclic carbonate is discharged from the reactor while the supported dimeric aluminium salen complex remains in the reactor. The liquid carbonate product is purified by means of distillation. Between the reactor and the distillation one or more buffer vessels are present having a volume of between 5 and 50 m³ per kmol of dimeric aluminium salen complex as present in the reactor.

Process to continuously prepare a cyclic carbonate product by reacting an epoxide compound with carbon dioxide in the presence of a heterogeneous catalyst which catalyst is activated by an activating compound and wherein the process is performed in at least a first, second, third reactor, each reactor comprising a slurry of the supported catalyst and the cyclic carbonate product as present as a liquid.

EP2257559B1 describes a continuous process to prepare ethylene carbonate from ethylene oxide and carbon dioxide is described. The reaction takes place in the presence of a dimeric aluminium salen complex supported on a modified SiO₂ support as the catalyst and nitrogen gas. The supported catalyst is present in a tubular reactor and the reactants are supplied to the tubular reactor as a gaseous mixture of ethylene oxide, carbon dioxide and nitrogen. The temperature in the reactor was kept at 60° C. by means of a water bath and the pressure was atmospheric. The yield of ethylene carbonate was 80%.

An advantage of the process of EP2257559B1 is that the reaction conditions may be close to ambient in terms of temperature and pressure. As a result of this the energy consumption of the process is low and less by-products are formed. A disadvantage however of the continuous process described in EP2257559B1 is that the tubular reactor requires external cooling to avoid overheating as a result of the exothermal reaction to ethylene carbonate.

WO2019/125151 describes a process where the carbon dioxide and the epoxide compound react in suspension of liquid cyclic carbonate and a supported dimeric aluminium salen complex. According to this publication the liquid cyclic carbonate product acts as an efficient heat transfer medium which avoids overheating. In this process the deactivated dimeric aluminium salen complex is reactivated by contacting the complex with a halide compound acting as an activating compound. The reactivation may be by adding the halide compound to the deactivated dimeric aluminium salen complex in a separate step while not adding extra carbon dioxide and epoxide compound. Purified cyclic carbonate is obtained in a distillation step wherein halide compound and other lower boiling compounds are separated from the cyclic carbonate. A problem is that after performing a reactivation and subsequently commencing the reaction between carbon dioxide and the epoxide compound a reactor effluent is obtained with high contents of the halide compound. This results in a step wise increase of the halide compound content in the feed to the downstream distillation step. This results in that the distillation step is difficult to operate.

The object of this invention is to provide a process which does not have the disadvantages as described for the process of WO2019/125151. This is achieved with the following process.

Process to continuously prepare a cyclic carbonate product by reacting an epoxide compound with carbon dioxide in the presence of a supported dimeric aluminium salen complex which catalyst is activated by a halide compound,

wherein the process is performed in a reactor comprising a slurry of the supported dimeric aluminium salen complex and the cyclic carbonate product as present as a liquid and wherein to the reactor carbon dioxide and the epoxide compound is continuously supplied and a liquid cyclic carbonate product stream comprising part of the halide compound and dissolved epoxide compound is discharged while substantially all of the supported dimeric aluminium salen complex remains in the reactor,

wherein the cyclic carbonate product as present in the liquid cyclic carbonate product stream is separated from the halide compound in a distillation step wherein a purified cyclic carbonate product is obtained as a bottom product of the distillation step and wherein between the reactor and the distillation step the liquid cyclic carbonate product stream passes a one or more buffer vessels, wherein the total volume of the one or more buffer vessels expressed in m³ relative to the amount of dimeric aluminium salen complex as present in the reactor and expressed in kmol is between 5 and 50 m³/kmol.

Applicants found that by performing the process according to the invention a more stable distillation process is achieved. It has been found that an optimal buffer volume relates to the amount of dimeric aluminium salen complex as present in the reactor. It is believed that in time a varying amount of halide compound will be released by the dimeric aluminium salen complex. This is especially the case when the process is performed in more than one reactor and wherein at least one reactor is regenerated at a time. When such a regenerated reactor is put on stream a high content of halide compound may be present in the liquid cyclic carbonate product stream. The present process allows one to use in all cycle steps of the process the same distillation equipment and achieve the desired high-purity in the cyclic carbonate product. The content of halide compound in the liquid cyclic carbonate product stream will then vary between a very low value and an even lower value. By passing the liquid cyclic carbonate product stream via a buffer vessel the content of the halide compound is more averaged and surprisingly it has been found that this results in a more stable operation of the distillation step.

The supported dimeric aluminium salen complex may be any supported complex as disclosed by the earlier referred to EP2257559B1. Preferably the complex is represented by the following formula:

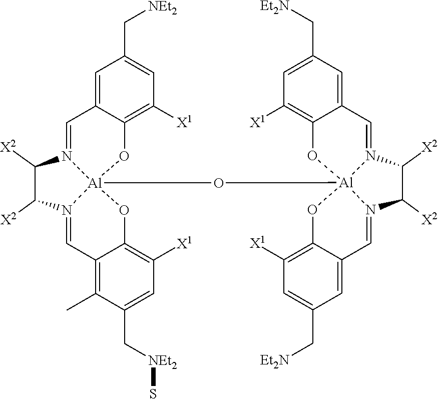

wherein S represents a solid support connected to the nitrogen atom via an alkylene bridging group, wherein the supported dimeric aluminium salen complex is activated by a halide compound. The alkylene bridging group may have between 1 and 5 carbon atoms. X² may be a C6 cyclic alkylene or benzylene. Preferably X² is hydrogen. X¹ is preferably a tertiary butyl. Et in the above formula represents any alkyl group, preferably having from 1 to 10 carbon atoms. Preferably Et is an ethyl group.

S represents a solid support. The catalyst complex may be connected to such a solid support by (a) covalent binding, (b) steric trapping or (c) electrostatic binding. For covalent binding, the solid support S needs to contain or be derivatized to contain reactive functionalities which can serve for covalently linking a compound to the surface thereof. Such materials are well known in the art and include, by way of example, silicon dioxide supports containing reactive Si—OH groups, polyacrylamide supports, polystyrene supports, polyethyleneglycol supports, and the like. A further example is sol-gel materials. Silica can be modified to include a 3-chloropropyloxy group by treatment with (3-chloropropyl)triethoxysilane. Another example is Al pillared clay, which can also be modified to include a 3-chloropropyloxy group by treatment with (3-chloropropyl)triethoxysilane. Solid supports for covalent binding of particular interest in the present invention include siliceous MCM-41 and MCM-48, optionally modified with 3-aminopropyl groups, ITQ-2 and amorphous silica, SBA-15 and hexagonal mesoporous silica. Also of particular interest are sol-gels. Other conventional forms may also be used. For steric trapping, the most suitable class of solid support is zeolites, which may be natural or modified. The pore size must be sufficiently small to trap the catalyst but sufficiently large to allow the passage of reactants and products to and from the catalyst. Suitable zeolites include zeolites X, Y and EMT as well as those which have been partially degraded to provide mesopores, that allow easier transport of reactants and products. For the electrostatic binding of the catalyst to a solid support, typical solid supports may include silica, Indian clay, Al-pillared clay, Al-MCM-41, K10, laponite, bentonite, and zinc-aluminium layered double hydroxide. Of these silica and montmorillonite clay are of particular interest. Preferably the support S is a particle chosen from the group consisting of silica, alumina, titania, siliceous MCM-41 or siliceous MCM-48.

Preferably the support S has the shape of a powder having dimensions which are small enough to create a high active catalytic surface per weight of the support and large enough to be easily separated from the cyclic carbonate in or external of the reactor. Preferably the support powder particles have for at least 90 wt % of the total particles a particle size of above 10 μm and below 2000 μm. The particle size is measured by a Malvern® Mastersizer® 2000.

The supported catalyst complex as shown above is activated by a halide compound. The halide compound will comprise a halogen atom which halogen atom may be CI, Br or I and preferably Br. The quaternary nitrogen atom of the complex shown above is paired with the halide counterion. Possible activating compounds are described in EP2257559B1 which exemplifies tetrabutylammonium bromide as a possible activating compound. Benzyl bromide is a preferred activating compound because it can be separated from the preferred cyclic carbonate product, such as propylene carbonate and ethylene carbonate by distillation.

An example of a preferred supported dimeric aluminium salen complex which complex is activated by benzyl bromide is shown below, wherein Et is ethyl and tBu is tert-butyl and Osilica represents a silica support:

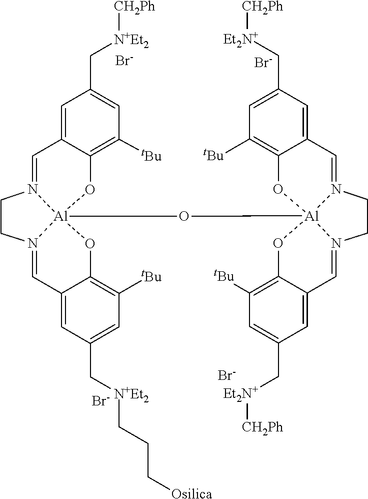

In use the Et group in the above formula may be exchanged with the organic group of the halide compound. For example if benzyl bromide is used as the halide compound to activate the above supported dimeric aluminium salen complex the Et group will be exchanged with the benzyl group when the catalyst is reactivated.

The epoxide product may be the epoxides as described in the afore mentioned EP2257559B1 in paragraphs 22-26. Preferably the epoxide compound has 2 to 8 carbon atoms. Preferred epoxide compounds are ethylene oxide, propylene oxide, butylene oxide, pentene oxide, glycidol and styrene oxide. The cyclic carbonate products which may be prepared from these preferred epoxides have the general formula:

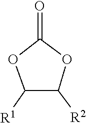

Where R¹ is a hydrogen or a group having 1-6 carbon atoms, preferably hydrogen, methyl, ethyl, propyl, hydroxymethyl and phenyl, and R² is hydrogen.

In the reactor the carbon dioxide is contacted with the epoxide compound in a suspension of liquid cyclic carbonate. The temperature and pressure conditions are chosen such that the cyclic carbonate is in its liquid state. The temperature and pressure conditions are further chosen such that carbon dioxide and epoxide easily dissolve in the liquid cyclic carbonate reaction medium. The temperature may be between 0 and 200° C. and the pressure is between 0 and 5.0 MPa (absolute) and wherein temperature is below the boiling temperature of the cyclic carbonate product at the chosen pressure. At the high end of these temperature and pressure ranges complex reactor vessels will be required. Because favourable results with respect to selectivity and yield to the desired carbonate product are achievable at lower temperatures and pressures it is preferred that the temperature in the first and second reactor is between 20 and 150° C., more preferably between 40 and 120° C., and the absolute pressure is between 0.1 and 0.5 MPa, more preferably between 0.1 and 0.3 MPa.

The liquid cyclic carbonate product stream as discharged from the reactor will comprise some epoxide compound and some activator compound as dissolved in the liquid product steam. This epoxide compound is suitably separated from the product stream and returned to the reactor. More preferably the dissolved epoxide as present in the liquid cyclic carbonate product stream is stripped out by contacting the liquid cyclic carbonate product stream with carbon dioxide resulting in a cleaned product stream and a loaded carbon dioxide stream containing epoxide compound and wherein the loaded carbon dioxide stream is supplied to the reactor.

The cleaned product stream may still contain some activator compound, carbon dioxide and epoxide compound. The content of activator compound in this stream may vary over time as explained above.

In the distillation step the cyclic carbonate product as present in the preferred cleaned product stream is separated from the activating compound and epoxide compound in the distillation step wherein a purified cyclic carbonate product is suitably obtained as a bottom product of the distillation step. The activating compound obtained as the top product in this distillation may be further purified by separation of any entrained gasses, such as carbon dioxide and epoxide compound. The activating compound obtained in the distillation step may be used to activate the deactivated catalyst. The activating compound may be directly added to the reactor and/or stored. The stored activator compound may then be added at another moment in time to the reactor.

The dimeric aluminium salen complex catalyst will deactivate over time. Activation of the catalyst is performed by contacting the deactivated catalyst with the halide compound. Halide compound may be added to the reactor in an amount sufficient to decrease deactivation of the supported dimeric aluminium salen complex. This addition may be performed while preparing the cyclic carbonate product in the reactor. Such a method of activation may result in a variation of the content of halide compound in the liquid cyclic carbonate product stream as discharged from the reactor. The activation of the dimeric aluminium salen complex catalyst may also be performed by adding the halide compound while no reactants, ie carbon dioxide and/or epoxide compound, are added to the reactor. When the dimeric aluminium salen complex catalyst is activated the flow of reactants to the reactor is again started to perform the preparation of the cyclic carbonate according to the process of this invention.

The reactor may be a single reactor or more than one reactor. When one reactor is used it is preferred to activate the dimeric aluminium salen complex catalyst by adding halide compound while preparing the cyclic carbonate product. Alternatively two reactors may be used wherein in one reactor the cyclic carbonate product is prepared and wherein in the other reactor the dimeric aluminium salen complex catalyst is activated. In an even more preferred configuration a further reactor as a second reactor is positioned in series with the reactor which becomes a first reactor. This second reactor comprises a slurry of the supported dimeric aluminium salen complex and the cyclic carbonate product as present as a liquid. A second liquid cyclic carbonate product stream comprising liquid cyclic carbonate product, part of the halide compound and dissolved epoxide compound is discharged. Substantially all of the supported dimeric aluminium salen complex remains in the second reactor. From the first reactor unreacted carbon dioxide and epoxide is discharged as a first gaseous effluent, which gaseous effluent is continuously supplied to the second reactor. The cyclic carbonate product as present in the second liquid cyclic carbonate product stream is separated from the halide compound in the distillation step. Between the second reactor and the distillation step the second liquid cyclic carbonate product stream passes one or more buffer vessels separately or in admixture with the first liquid cyclic carbonate product stream. The total volume of the one or more buffer vessels expressed in m³ relative to the amount of dimeric aluminium salen complex as present in the first and second reactor and expressed in kmol is between 5 and 50 m³/kmol. When the first and second reactor are operated in a series mode as described above a higher conversion of the epoxide compound is achieved.

In such a two reactor in series mode as described above unreacted carbon dioxide and epoxide may be discharged as a second gaseous effluent from the second reactor. Suitably part of the second gaseous effluent is recycled to the first reactor and part of the second gaseous effluent is purged from the process.

In the two reactors in series mode as described above it is preferred that in a cycle step of the process a deactivated supported dimeric aluminium salen complex as present in a third reactor as a slurry of the supported dimeric aluminium salen complex and the cyclic carbonate product as present as a liquid is activated by adding halide compound. No reactants, ie carbon dioxide and/or epoxide compound, are added to the third reactor. After activating the dimeric aluminium salen complex in such an off-line reactor a next cycle step of the process is performed wherein the third reactor becomes the second reactor, the second reactor becomes the first reactor and the first reactor becomes the third reactor. Preferably the time period of one cycle step of the process is between 1-30 days, preferably between 2-20 days. In such a period of time cyclic carbonate product may be continuously be prepared in the first and second reactor. The addition of the activating compound to the third reactor to obtain a reactor comprising activated heterogeneous catalyst may be performed in a shorter time period.

In the two reactors in series mode the first, second and third reactor change their relative operating mode after each cycle step of the process. One cycle step of the process involves operating the first and second reactor as described to prepare the cyclic carbonate product while the catalyst in the third reactor is regenerated. At the start of a next cycle step the third reactor becomes the second reactor, the second reactor becomes the first reactor and the first reactor becomes the third reactor. This may be achieved by operating a set of sequence valves which result in that the previous third reactor is connected to the previous second reactor in such a way that these reactors will operate as the first and second reactor according to the invention. The previous first reactor, comprising deactivated heterogeneous catalyst, is disconnected from the supply conduits for carbon dioxide and epoxide compound and connected to a supply conduit for the activating compound.

The process may be performed in more than the 3 reactors described above, referred to as a reactor train. For example more than one reactor train may be operated in parallel according to the process of this invention. The reactor effluents of these trains may be separated into products and activating compounds in a common separation process, ie distillation step.

A reactor train may comprise a further reactor, referenced as the intermediate reactor. The intermediate reactor comprises a slurry of the heterogeneous catalyst and the cyclic carbonate product as present as a liquid similar to the other reactors. To the intermediate reactor the gaseous effluent of an upstream reactor in the reactor train is continuously supplied, liquid cyclic carbonate is discharged as an intermediate reactor product stream and unreacted carbon dioxide and epoxide is discharged as an intermediate reactor gaseous effluent stream. Substantially all of the heterogeneous catalyst remains in the intermediate reactor. A train with more than 3 reactors will comprise of the first, second and third reactor according to the process of the invention. The additional reactors will operate in series with the first and second reactor, wherein the additional reactors will be placed between first and second reactor.

In a reactor train the gaseous effluent as discharged from a reactor will be supplied to a downflow reactor. The pressure in a reactor in a train of reactors may be higher than the pressure in the next downflow reactor. This is advantageous because no special measures, such as compressor or blowers, have to be present to create a flow of the gaseous effluent to the next downflow reactor. Suitably part gaseous effluent of the most downflow reactor in a train of reactors is recycled to the first reactor and part of this gaseous effluent is purged from the process. In the two reactors in series mode suitably part of the second gaseous effluent is recycled to the first reactor and part of the second gaseous effluent is purged from the process.

The reactors may be any reactor in which the reactants and catalyst in the liquid reaction mixture can intimately contact and wherein the feedstock can be easily supplied to. The reactor is suitably a continuously operated reactor. To such a reactor carbon dioxide and the epoxide compound is continuously supplied and liquid cyclic carbonate is discharged. The speed at which the gaseous carbon dioxide and the gaseous or liquid epoxide is supplied could agitate the liquid contents of the reactor such that a substantially evenly distributed reaction mixture results. Sparger nozzle may be used to add a gaseous compound to the reactor. Such agitation may also be achieved by using for example ejectors or mechanical stirring means, like for example impellers. Such reactors may be of the so-called bubble column slurry type reactor and mechanically agitated stirred tank reactor. In a preferred embodiment the reactor is a continuously operated stirred reactor wherein carbon dioxide and epoxide compound are continuously supplied to the reactor and wherein part of the cyclic carbonate product is continuously withdrawn as part of a liquid stream. The reactors of a reactor train are preferably of the same size and design. The reactors of parallel operated reactor trains may be different for each train.

In the process of the invention substantially all of the heterogeneous catalyst remains in the reactor while part of the liquid cyclic carbonate product is discharged from the reactor. Preferably a volume of liquid cyclic carbonate product is discharged from the reactor which corresponds with the production of cyclic carbonate product in the reactor such that the volume of suspension in the reactor remains substantially the same in time. The liquid cyclic carbonate is separated from the heterogeneous catalyst by a filter. This filter may be positioned external of the reactor. Preferably the filter is positioned within the reactor. A vertical positioned cylindrical vessel is preferred. A preferred filter is a cross-flow filter. For the preferred supported dimeric aluminium salen complex as the catalyst is a 10 μm filter, more preferably composed of a so-called Johnson Screens® using Vee-Wire® filter elements, is preferred. The filter may have the shape of a tube placed vertically in the reactor, preferably in the preferred vertical positioned cylindrical vessel. The filter may be provided with means to create a negative flow over the filter such to remove any solids from the filter opening.

Preferably all or part of the epoxide as obtained in the distillation is directly or indirectly recycled to the first reactor. In this way all or almost all of the epoxide can be converted to the cyclic carbonate product. Part of the epoxide as obtained in the distillation may be purged such to avoid a build-up of compounds boiling in the same range as the epoxide. These other compounds may have been present in any one of the feedstocks or which may have formed in the process.

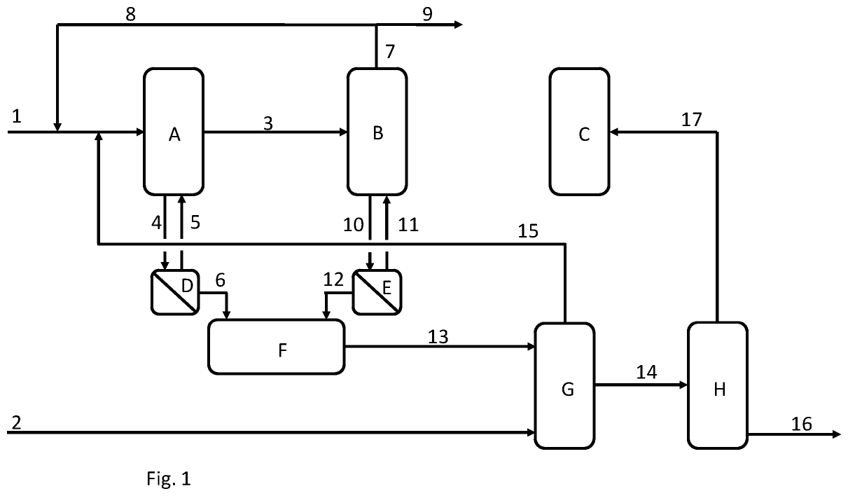

The invention shall be illustrated by FIG. 1. FIG. 1 shows a flow scheme of the process according to the invention starting from propylene oxide and using a supported dimeric aluminium salen complex as activated by benzyl bromide as the catalyst. A first reactor (A), a second reactor (B) and a third reactor (C) is shown. All three reactors comprise of a slurry of the catalyst and the cyclic carbonate product. To the first reactor (A) carbon dioxide is continuously supplied via stream (2), stripper (G) and stream (15). In stripper (G) the carbon dioxide gas contacts a combined liquid product stream (13) to obtain a cleaned liquid product stream (14) and a loaded carbon dioxide stream (15) containing some propylene oxide compound. This stream (15) is combined with fresh propylene oxide as supplied via (1) and part of the unreacted carbon dioxide and propylene oxide from the second reactor (8), and the combined stream is supplied to the first reactor (A). In reactor (A) a liquid cyclic carbonate is formed by reaction of carbon dioxide and propylene oxide. During the process step part of the slurry is discharged as stream (4) to a filter (D). In this filter liquid cyclic carbonate is separated from the catalyst. The catalyst is returned to reactor (A) via stream (5) and liquid cyclic carbonate poor in catalyst discharged as a first product stream (6) from reactor (A). Unreacted carbon dioxide and propylene oxide is discharged as a first gaseous effluent stream (3) and continuously supplied to second reactor (B).

In reactor (B) a liquid cyclic carbonate is formed by reaction of carbon dioxide and propylene oxide. During the process step part of the slurry is discharged as stream (10) to a filter (E). In this filter liquid cyclic carbonate is separated from the catalyst. The catalyst is returned to reactor (B) via stream (11) and liquid cyclic carbonate poor in catalyst discharged as a second product stream (12) from reactor (B). Unreacted carbon dioxide and propylene oxide is discharged as a second gaseous effluent stream (7) of which part is recycled to first reactor (A) and part is purged via stream (9).

Cleaned product streams (6) and (12) are collected in a buffer vessel (F). From this buffer vessel (F) a combined product stream (13) is fed to the stripper (G). The cleaned liquid product stream (14) is fed to a distillation column (H) wherein the cyclic carbonate product as present in cleaned product stream is separated from the benzyl bromide and other lower boiling compounds. The benzyl bromide is fed via stream (17) to the third reactor (C), optionally via a storage vessel (not shown). A purified cyclic carbonate product is obtained as a bottom product (16) in the distillation column (H).

In the process step the deactivated supported dimeric aluminium salen complex as present in the third reactor (C) is activated by adding benzyl bromide via stream (17). The process step ends when the catalyst is re-activated and/or when the catalyst activity in the combined first reactor (A) and second reactor (B) drops below an unacceptable level. A next step starts by switching the reactors such that the third reactor (C) becomes the second reactor (B′), the second reactor (B) becomes the first reactor (A′) and the first reactor (A) becomes the third reactor (C′). In this way the deactivated catalyst of first reactor (A) at the end of the previous step can be activated.

The invention will be illustrated by the following non-limiting example.

### EXAMPLE

Reference is made to the process shown in FIG. 1. The concentration of benzyl bromide in stream (13) of FIG. 1 is calculated for a three reactor configuration as shown wherein the production of propylene carbonate is kept at a constant value. The total amount of dimeric aluminium salen complex as present in the operating reactors (A) and (B) is 0.22 kmol. During a step between 175 and 213 kg/hr of carbon dioxide is fed to first reactor (A) and between 154 and 187 kg/hr of propylene oxide is fed to first reactor (A). These values change due to the deactivation of the catalyst in reactors (A) and (B) and because the production of propylene carbonate is kept at the same level.

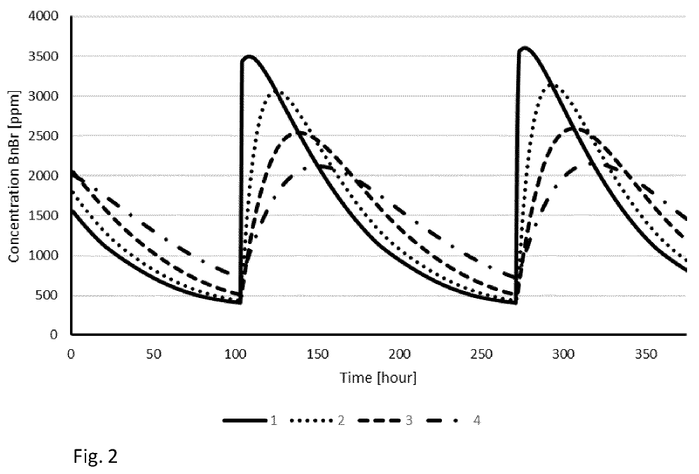

In a first simulation of the process no buffer vessel (F) is present. In a second simulation of the process a buffer vessel (F) is present having a volume of 2 m³. In a third simulation of the process a buffer vessel (F) is present having a volume of 5 m³. In a fourth simulation of the process a buffer vessel (F) is present having a volume of 10 m³. The effect of the presence of a buffer vessel (F) and its size is illustrated in FIG. 2. It is shown that by having a buffer vessel (F) the maximum and also minimum benzyl bromide contents in this stream (13) will be less extreme making it easier to perform the downstream distillation (H).

1. A process to continuously prepare a cyclic carbonate product by reacting an epoxide compound with carbon dioxide in the presence of a supported dimeric aluminium salen complex which catalyst is activated by a halide compound,
wherein the process is performed in a reactor comprising a slurry of the supported dimeric aluminium salen complex and the cyclic carbonate product as present as a liquid and wherein to the reactor carbon dioxide and the epoxide compound is continuously supplied and a liquid cyclic carbonate product stream comprising part of the halide compound and dissolved epoxide compound is discharged while substantially all of the supported dimeric aluminium salen complex remains in the reactor,
wherein the cyclic carbonate product as present in the liquid cyclic carbonate product stream is separated from the halide compound in a distillation step wherein a purified cyclic carbonate product is obtained as a bottom product of the distillation step and wherein between the reactor and the distillation step the liquid cyclic carbonate product stream passes one or more buffer vessels,
wherein the total volume of the one or more buffer vessels expressed in m³ relative to the amount of dimeric aluminium salen complex as present in the reactor and expressed in kmol is between 5 and 50 m³/kmol.

2. The process according to claim 1, wherein the supported dimeric aluminium salen complex is represented by the following formula: 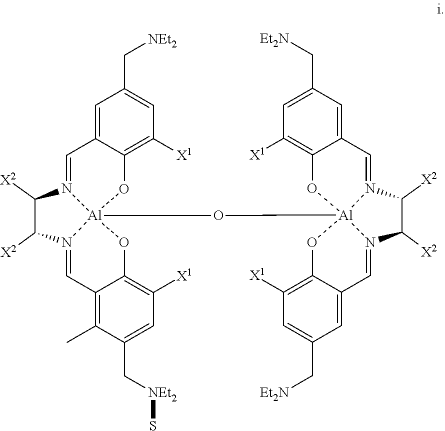
wherein S represents a solid support connected to the nitrogen atom via an alkylene group, wherein the supported dimeric aluminium salen complex is activated by a halide compound and wherein X¹ is tertiary butyl and X² is hydrogen and wherein Et is an alkyl group having 1 to 10 carbon atoms.

3. The process according to claim 2, wherein the support S is composed of particles having an average diameter of between 10 and 2000 μm.

4. The process according to claim 3, wherein the support S is a particle chosen from the group consisting of silica, alumina, titania, siliceous MCM-41 or siliceous MCM-48.

5. The process according to claim 1, wherein the halide compound is benzyl halide.

6. The process according to claim 5, wherein the benzyl halide is benzyl bromide.

7. The process according to claim 1, wherein the epoxide compound has 2 to 8 carbon atoms.

8. The process according to claim 7, wherein the epoxide compound is ethylene oxide, propylene oxide, butylene oxide, pentene oxide, glycidol or styrene oxide.

9. The process according to claim 1, wherein the temperature in the reactor is between 20 and 150° C. and the absolute pressure is between 0.1 and 0.5 MPa and wherein temperature is below the boiling temperature of the cyclic carbonate product at the chosen pressure.

10. The process according to claim 1, wherein the dissolved epoxide as present in the liquid cyclic carbonate product stream is stripped out by contacting the liquid cyclic carbonate product stream with carbon dioxide resulting in a cleaned product stream and a loaded carbon dioxide stream containing epoxide compound and wherein the loaded carbon dioxide stream is supplied to the reactor.

11. The process according to claim 1, wherein to the reactor halide compound is added in an amount sufficient to decrease deactivation of the supported dimeric aluminium salen complex.

12. The process according to claim 1,
wherein a further reactor as a second reactor is positioned in series with the reactor which becomes a first reactor,
wherein the second reactor comprises a slurry of the supported dimeric aluminium salen complex and the cyclic carbonate product as present as a liquid, and wherein a second liquid cyclic carbonate product stream comprising liquid cyclic carbonate product, part of the halide compound and dissolved epoxide compound is discharged while substantially all of the supported dimeric aluminium salen complex remains in the second reactor,
wherein from the first reactor unreacted carbon dioxide and epoxide is discharged as a first gaseous effluent, which gaseous effluent is continuously supplied to the second reactor, and
wherein the cyclic carbonate product as present in the second liquid cyclic carbonate product stream is separated from the halide compound in the distillation step and wherein between the second reactor and the distillation step the second liquid cyclic carbonate product stream passes one or more buffer vessels separately or in admixture with the first liquid cyclic carbonate product stream, wherein the total volume of the one or more buffer vessels expressed in m³ relative to the amount of dimeric aluminium salen complex as present in the first and second reactor and expressed in kmol is between 5 and 50 m³/kmol.

13. The process according to claim 12, wherein in a cycle step of the process a deactivated supported dimeric aluminium salen complex as present in a third reactor as a slurry of the supported dimeric aluminium salen complex and the cyclic carbonate product as present as a liquid is activated by adding halide compound and wherein in a next cycle step of the process the third reactor becomes the second reactor, the second reactor becomes the first reactor and the first reactor becomes the third reactor.

14. The process according to claim 13, wherein the time period of one cycle step of the process is between 1-30 days.

15. The process according to claim 11, wherein the added halide compound is obtained in the distillation step.

16. The process according to claim 12, wherein from the second reactor unreacted carbon dioxide and epoxide is discharged as a second gaseous effluent and wherein part of the second gaseous effluent is recycled to the first reactor and part of the second gaseous effluent is purged from the process.
